(19) United States
(12) Patent Application Publication
(10) Pub. No.: US 2026/0114746 A1
(43) Pub. Date: Apr. 30, 2026

(54) SYSTEM AND METHOD OF PREDICTING AND DIAGNOSING OSTEOPOROSIS ON THE BASIS OF MAGNETIC RESONANCE IMAGING TECHNIQUE

(71) Applicant: M-SCORE BONE HEALTH S.R.L., Crema (CR) (IT)

(72) Inventors: Tiberio Dario Maria Bozotti, Crema (CR) (IT); Michele Bandirali, Crema (CR) (IT); Luigi Olmo, Crema (CR) (IT)

(73) Assignee: M-SCORE BONE HEALTH S.R.L., Crema (CR) (IT)

(21) Appl. No.: 19/480,419

(22) PCT Filed: May 2, 2024
(86) PCT No.: PCT/IB2024/054252

§ 371 Date: Oct. 31, 2025

(30) Foreign Application Priority Data
May 3, 2023 (IT) 102023000008730

(51) Int. Cl. A61B 5/055; A61B 5/00

(52) U.S. Cl. CPC A61B 5/055; A61B 5/4509; A61B 5/4566; A61B 5/7267; A61B 5/7275

(57) ABSTRACT

It is disclosed a computer-implemented system and method of predicting and diagnosing osteoporosis on the basis of magnetic resonance imaging techniques. Particularly, the method of the invention is a reliable, reproducible and sensitive method for predicting or diagnosing osteoporosis from magnetic resonance images, through a simple and quick statistical processing, i.e. by only statistically correlating recorded MRI of a subject to recorded MRI of reference groups, independently of other standard techniques such as, for example, MOC, QCT, DXA, or clinical diagnosis of osteoporosis. In this regard, any patient who undergoes magnetic resonance imaging for reasons independent of osteoporosis, could anyway receive diagnostic information also on his/her medical status concerning osteoporosis, without additional investigations or tests and without the use of ionizing radiation, regardless of previous suspicion of osteoporotic pathology. This is very precious and desirable as, in this way, it is possible to early identify patients at risk of osteoporosis or with the pathology already in progress, notwithstanding the asymptomatic nature of osteoporosis. Moreover, the overall method of the invention is very convenient insofar as neither the patient nor the national health system incur additional costs.

### FIELD OF THE INVENTION

The present invention concerns a computer-implemented method of predicting and diagnosing osteoporosis on the basis of magnetic resonance imaging techniques. Particularly, the method of the invention allows to give a quick and reliable outcome by simply elaborating the image resulting from a magnetic resonance, preferably a lumbar spine magnetic resonance, and comparing the same with a pre-acquired database. In this regard, the present invention also concerns a computer-implemented system for executing the above method.

The present invention also concerns a computer program comprising instructions which, when the program is executed by a computer, cause the computer to carry out the steps of the above method, as well as relates to a computer-readable data carrier having stored thereon said computer program.

### STATE OF THE ART

Osteoporosis (‘OP’) is characterized by a systemic reduction in bone mass leading to increased bone fragility and an increased risk of bone fracture. Osteoporosis is an asymptomatic disease, which means that patients do not exhibit clear symptoms at the onset or during progression. Consequently, the diagnosis of osteoporosis frequently is made after a patient has sustained a fragility (osteoporotic) fracture. Osteoporotic fractures occur most frequently at the femoral neck, hip, spine or forearm. In those cases, the term “established osteoporosis” is used, i.e. when a broken bone due to osteoporosis has occurred.

Osteoporotic fractures are caused by an increase in bone fragility, which can occur due to low bone mass and microarchitectural changes in bone tissue. With a lifetime risk of 30%-40% to be affected by vertebral or non-vertebral fractures in developed countries, osteoporosis has an incidence rate similar to that of coronary heart disease. Furthermore, with the exception of forearm fractures, osteoporotic fractures are associated with increased mortality. Most fractures cause acute pain and lead to patient hospitalization, immobilization and often slow recovery. In addition, osteoporotic symptoms are frequently observed in patients with type 2 diabetes, who overall suffer from an elevated risk of fragility fractures. Diabetes mellitus refers to a group of metabolic diseases in which a subject has high blood sugar. Type 2 diabetes results from insulin resistance, a condition in which cells fail to use insulin properly, sometimes also with an absolute insulin deficiency. This form was previously referred to as non insulin-dependent diabetes mellitus (NIDDM) or “adult-onset diabetes”.

Accurate tools for early prediction, diagnosis and fracture-risk assessment are therefore critical to target the right method of treatment to the OP patients in need thereof.

Current methods for the assessment of fracture risk as well as treatment response include non-invasive imaging techniques as well as the analysis of clinical parameters and biochemical markers of bone turnover.

The most currently used techniques for diagnosing osteoporosis are CBM (i.e. Computerized Bone Mineralometry), QCT (i.e. Quantitative Computer Tomography), and DXA (i.e. Dual energy X-ray absorptiometry).

In particular, CBM is a diagnostic technique that evaluates bone mineralization. This technique uses the penetration of X-rays or ultrasound to express the bone mass density (BMD) as T-score or Z-score. In more details, T-score expresses the difference in number of standard deviations between the patient's individual value and the average value of the healthy reference population, whereas Z-score expresses the difference between the patient's value and the value of a healthy reference population consisting of subjects of the same gender and age. The BMD value reveals the state of bone degeneration and gives prognostic and diagnostic indications on osteoporosis.

QCT is a medical technique that measures BMD by using a standard X-ray Computed Tomography (CT) scanner with a calibration standard to convert Hounsfield Units (HU) of the CT image to BMD values. Quantitative CT scans are primarily used to evaluate bone mineral density at the lumbar spine and hip.

DXA is a well standardized and easy-to-use quantitative imaging modality that has high precision (maximum acceptable precision error 2%-2.5%) and uses a nearly negligible radiation dose. In 1994, BMD testing with DXA has been adopted as a standard of reference in managing osteoporosis using the T-score to classify BMD.

In the prophylaxis, diagnosis and management of osteoporosis, the assessment of fracture risk and monitoring of treatment response are two of the most important aspects. Therefore, analysis of bone mass by measuring bone mineral density (BMD) is currently the only clinical parameter of the skeleton that is routinely analysed in clinical practice and part of the WHO FRAX questionnaire (Kanis et al., 2013).

However, as osteoporosis is a “silent disease”, a subject has typically no reasons to undergo to said examination techniques, absent any symptom prompting the subject himself/herself to check his/her own medical situation. This exposes the subject to risk of fractures without having the possibility to prevent them by early investigation.

It is therefore felt the need of a reliable method for an early prediction and diagnosis of osteoporosis, which overcome the drawbacks of the prior art and gives significant results that the patients can attest and appreciate.

### SUMMARY OF THE INVENTION

This and other objects are achieved through a computer-implemented system and method of predicting and diagnosing osteoporosis on the basis of lumbar magnetic resonance imaging techniques, as defined in the claims.

As it will be apparent from the following detailed description, the method of the invention allows to reliably predict the risk of developing or diagnose osteoporosis by starting from a simple recorded magnetic resonance imaging (shortly ‘MRI’) of the lumbar spine, irrespectively of the reason why said MRI has been prescribed to a subject.

The method is based on the acquisition of the recorded lumbar spine MRI output, which is then analysed and statistically processed to give a quick and reliable result on the risk of developing osteoporosis (i.e. osteopenia) or on assessment of ongoing osteoporosis, as well as on assessment of established osteoporosis.

The method of the invention is therefore advantageously independently of any other known technique used as a reference standard such as, for example, CBM, DXA, QCT, or clinical diagnosis of osteoporosis.

Moreover, the method of the invention has resulted in a very rapid and efficient service of a only few steps for the medical operators, thus making the overall predictive and diagnostic process very convenient under both the economical and operative points of view.

The present invention also relates to a computer program comprising instructions which, when the program is executed by a computer, cause the computer to carry out the steps of the method of the invention.

Moreover, the present invention relates to a computer-readable data carrier having stored thereon said computer program.

### BRIEF DESCRIPTION OF THE DRAWINGS

The characteristics and advantages of the invention will be clear from the following detailed description of the invention, the working examples provided for illustrative and non-limiting purposes, as well as the accompanying figures, wherein:

FIG. 1 depicts an exemplary sagittal T1-weighted magnetic resonance image of the lumbar spine of a reference subject, elaborated according to the procedure reported in the Example 1,

FIG. 2 depicts an exemplary visual depiction of the calculation of M-score for a subject of Reference Group 2, as obtained according to the procedure reported in the Example 1,

FIG. 3 depicts the condition detected for the subjects of interest of Examples 1-3, according to step V-i) of the method of the invention,

FIG. 4 depicts an overview of the elements of the computer-implemented system of the invention, in an exemplified version according to Example 4, and

FIG. 5 shows an exemplary organization of data within the database according to Example 4.

### DETAILED DESCRIPTION OF THE INVENTION

The present invention therefore relates to a computer-implemented method of predicting and diagnosing osteoporosis on the basis of lumbar magnetic resonance imaging techniques, the method comprising the steps of:
I) collecting recorded lumbar spine MRI outputs, obtained from Magnetic Resonance apparatus, by means of a medical workflow management system selected from RIS, PACS, DICOM and CIS, and archiving to create a reference database, wherein said lumbar spine MRI outputs are classified into three different reference groups, according to the following criteria:
   Reference Group 1 (RG1)—MRI outputs of subjects aged 20-30 years
   Reference Group 2 (RG2)—MRI outputs of subjects aged higher than 75 years
   Reference Group 3 (RG3)—MRI outputs of subjects that have received a diagnosis of established osteoporosis, after fragility fracture detection within the lumbar spine area,
II) calculating the M-score for each MRI output of step I), according to the formula:

$$M ‐ score = ( S N {R}_{L 1 - L 4} - S N {R}_{R E F} ) / {SD}_{REF} \qquad ( A )$$

$$or$$

$$M ‐ score = ( SQ {R}_{L 1 - L 4} - S Q {R}_{R E F} ) / {SD}_{REF} \qquad ( B )$$

wherein
SNR_{L1-L4} denotes the signal-to-noise ratio obtained by dividing the intra-vertebral signal intensity by the standard deviation of the noise in vertebrae from L1 to L4 within the sagittal T1-weighted spin-echo sequence, of the MRI output,
SNR_{REF} denotes the SNR mean value of the Reference Group to which the MRI output belongs,
SQR_{L1-L4} denotes the signal-to-liquor ratio obtained by dividing the intra-vertebral signal intensity by the standard deviation of the liquor in vertebrae from L1 to L4 within the sagittal T1-weighted spin-echo sequence, of the MRI output,
SQR_{REF} denotes the SQR mean value of the Reference Group to which the MRI output belongs, and
SD_{REF} denotes the standard deviation of the Reference Group to which the MRI output belongs,
and then calculating the M-score mean value for each Reference Group of step I), named M-score(RG1), M-score(RG2), and M-score(RG3) respectively,
III) examining the recorded lumbar spine MRI output of a subject of interest (‘SOI’), by analysing the sagittal T1-weighted spin-echo sequence in vertebrae from L1 to L4, thus obtaining the relevant SNR_{L1-L4} (‘SOI-SNR_{L1-L4}’) or SQR_{L1-L4} (‘SOI-SQR_{L1-L4}’),
IV) calculating the M-score of the subject of interest with respect to each Reference Group of step I), according to the following formulas:

$$\begin{matrix}M ‐ score ( 1 ) = [ ( S O I - S N {R}_{L 1 - L 4} ) - S N {R}_{R E F} ( R G 1 ) ] / {SD}_{R E F} ( R G 1 ) \\ M ‐ score ( 2 ) = [ ( S OI - SN {R}_{L 1 - L 4} ) - S N {R}_{R E F} ( R G 2 ) ] / {SD}_{R E F} ( R G 2 ) \\ M ‐ score ( 3 ) = [ ( S OI - SN {R}_{L 1 - L 4} ) - S N {R}_{R E F} ( R G 3 ) ] / {SD}_{R E F} ( R G 3 )\end{matrix}$$

$$or$$

$$\begin{matrix}M ‐ score ( 1 ) = [ ( S OI - {SQR}_{L 1 - L 4} ) - S Q {R}_{R E F} ( R G 1 ) ] / {SD}_{R E F} ( R G 1 ) \\ M ‐ score ( 2 ) = [ ( S OI - {SQR}_{L 1 - L 4} ) - S Q {R}_{R E F} ( R G 2 ) ] / {SD}_{R E F} ( R G 2 ) \\ M ‐ score ( 3 ) = [ ( S OI - {SQR}_{L 1 - L 4} ) - S Q {R}_{R E F} ( R G 3 ) ] / {SD}_{R E F} ( R G 3 )\end{matrix}$$

and
V) comparing the resulting three M-scores of step IV) with the M-score mean value of the respective Reference Group resulting from step II), thus assessing for the subject of interest the possible risk of developing osteoporosis or diagnosing osteoporosis.

Actually, the steps I)-V) are implemented by a computer executing a computer program comprising instructions, which cause the computer to carry out said steps.

In step I) of the method, lumbar spine MRI outputs obtained from MR apparatus are collected and archived. With the term “lumbar spine MRI outputs”, it is meant to include any lumbar spine MRI output obtained from a subject medical investigation, irrespective of the reason underlying the initial prescription of MR, and recorded in digital folders along with other medical investigation outputs. A medical workflow management system supports and assists in storing, sharing and providing said recorded MRI outputs for the purposes of creating a reference database. Advantageously, this means that recorded lumbar spine MRI outputs of step I) are documents already available and retrieved from radiology archives of a clinic or a hospital.

In this regard, it should be noted that, within the healthcare environment, there are several medical workflow management systems that are employed on a daily basis. Many, if not all, are frequently found in radiology practices and departments. Four of the most leveraged radiology systems include RIS (Radiology Information System), PACS (Picture Archiving and Communication System), DICOM (Digital Imaging and Communications in Medicine), and CIS (Clinical Information System).

In more details, RIS is a root system used for electronic management by imaging departments. A RIS is a radiology-specific software solution geared to procuring, storing, and sharing medical imaging data and is used extensively throughout the healthcare sector. Designed to optimize efficiency of radiology workflows, a RIS can be used in conjunction with a Hospital Information System (HIS) and/or Picture Archiving and Communication System (PACS).

PACS is a medical imaging technology that provides data storage and convenient image access from multiple modalities. It is a common software solution used predominantly by hospitals and healthcare organizations. Built for to securely store and transmit electronic patient images and data, a PACS system eliminates the need for traditional methodologies involving manual file management (e.g. film jackets) and delivery. Though radiologists have been the main users of PACS systems, as well as being leading generators of x-ray images, PACS has also been implemented into other health-related fields including cardiology, oncology, dermatology, pathology, and nuclear medicine imaging. In light of its prolific usage, a PACS is designed to handle image formats produced from a wide variety of modalities such as mammograms (MG), magnetic resonance (MR), ultrasound (US), computed tomography (CT), and digital radiography. By leveraging a PACS, clinicians can easily access their patient information digitally. Digital access signifies accelerated and improve care, minimized chances of treatment and prescription errors, in addition to the circumvention of unnecessary testing performed.

DICOM, a worldwide-recognized standard protocol for the administration and transmission of medical images and data, is responsible for the present-day application of PACS. Actually, DICOM is the globally recognized communication and management standard for medical images and other patient data. It is frequently utilized throughout the medical field to store and transmit medical images facilitating integration with medical devices and PACS systems.

The difference between PACS and DICOM is that PACS is a medical image storage and archive hub, fed by medical modalities such as X-ray scanners and MRI machines, while DICOM represents the international communication standard—not a device or structure—used by healthcare professionals in storing, processing, transmitting medical images and data.

CIS involves networked software solutions working together within a radiology practice such as a RIS and electronic health record system. Geared specifically towards clinical care usage (e.g., Intensive Care Units), a CIS is an information system networks with computer systems utilized in various departments present-day hospitals. These departments include cardiology, radiology, and pathology. A CIS gathers patient data and transfers it into an electronic record which an attending clinician can access while visiting the patient's bedside.

For the purposes of the present invention, the creation of a reference database of MRI outputs can be assisted by any one of the systems above indicated, even if PACS is the most frequently used system to this end.

The reference database of step I) comprises lumbar spine MRI outputs obtained from MR apparatus. Preferably, said MRI outputs are obtained from the same MR apparatus model (i.e. different apparatus but of the same model). This provision allows to gather MRI outputs homogeneously obtained under the instrumental point of view. Actually, the MRI outputs can be obtained from different MR apparatus models however, understandably, if the same model is used, the statistically precision rate of the subsequent calculation of M-scores increases accordingly.

More preferably, the lumbar spine MRI outputs of step I) are obtained from the same MR apparatus. This means that all the lumbar spine MRI outputs are homogeneously obtained by using the same instrument, thus maximising the overall precision of the method.

In principle, under the statistical perspective, the higher the number of available MRI outputs, the lower the impact on the overall precision of the method, when MRI outputs derived from different MR apparatus models. Accordingly, the more homogeneous the source of MRI outputs (e.g. the same MS apparatus model, or better exactly the same MR apparatus), the lower the number of MRI outputs necessary to give a satisfactory precision of the method.

Step I) also includes the classification of said lumbar spine MRI outputs into three different reference groups, according to the following criteria:
Reference Group 1 (RG1)—MRI outputs of subjects aged 20-30 years
Reference Group 2 (RG2)—MRI outputs of subjects aged higher than 75 years
Reference Group 3 (RG3)—MRI outputs of subjects that have received a diagnosis of
established osteoporosis, after fragility fracture detection within the lumbar spine area.

RG1 gathers the MRI outputs of subjects aged 20-30 years, who are typically and statistically healthy with respect to osteoporosis. Therefore, this is referred to as the ‘healthy population’ group.

RG2 gathers the MRI outputs of subjects aged higher than 75 years, who are typically and statistically at high risk of developing osteoporosis or currently affected by osteoporosis. Therefore, this is referred to as the ‘age-related risk population’ group.

RG3 gathers the MRI outputs of subjects that have received a diagnosis of established osteoporosis, after fragility fracture detection within the lumbar spine area. Therefore, this is referred to as the ‘OP patients population’ group.

In step II) of the method, the M-score for each MRI output of step I) is calculated according to the formula:

$$M ‐ score = ( S N {R}_{L 1 - L 4} - S N {R}_{R E F} ) / {SD}_{REF} \qquad ( A )$$

$$or$$

$$M ‐ score = ( SQ {R}_{L 1 - L 4} - S Q {R}_{R E F} ) / {SD}_{REF} \qquad ( B )$$

wherein
SNR_{L1-L4} denotes the signal-to-noise ratio obtained by dividing the intra-vertebral signal intensity by the standard deviation of the noise in vertebrae from L1 to L4 within the sagittal T1-weighted spin-echo sequence, of the MRI output,
SNR_{RFF} denotes the SNR mean value of the Reference Group to which the MRI output belongs,
SQR_{L1-L4} denotes the signal-to-liquor ratio obtained by dividing the intra-vertebral signal intensity by the standard deviation of the liquor in vertebrae from L1 to L4 within the sagittal T1-weighted spin-echo sequence, of the MRI output,
SQR_{REF} denotes the SQR mean value of the Reference Group to which the MRI output belongs, and
SD_{REF} denotes the standard deviation of the Reference Group to which the MRI output belongs,
and then calculating the M-score mean value for each Reference Group of step I), named M-score(RG1), M-score(RG2), and M-score(RG3) respectively.

In particular, the imaging analysis protocol included, for all the MRI outputs of the method, a sagittal T1-weighted spin-echo sequence, optimal for evaluating vertebral fatty marrow, while segmenting vertebral bodies from L1 to L4. A region of interest (ROI) was placed in the vertebral body excluding cortical bone, subchondral abnormalities, focal lesions (e.g., hemangiomas), and posterior venous plexus. Three ROIs were used for each vertebra, each of them acquired on a different slice, with their mean used for analysis. A ROI was also positioned in an artefact-free site outside the patient to measure the noise. Signal-to-noise ratio (SNR) was obtained by dividing the intra-vertebral signal intensity by the standard deviation of the noise. The positioning of the ROIs can be done manually by an operator using the functions of any processing system (such as PACS, RIS or DICOM), or preferably by a semi-automatic or automatic system possibly able to be implemented with the use of artificial intelligence with a machine learning system.

The SNR_{L1-L4} was measured in all MRI outputs of the each reference group, then the respective mean value (SNR_{REF}) and standard deviation (SD_{REF}) were calculated; subsequently, according to formula A, the M-score mean value for each Reference Group is calculated, i.e. M-score(RG1), M-score(RG2), and M-score(RG3), respectively.

Alternatively and analogously, the M-score mean value for each Reference Group, i.e. M-score(RG1), M-score(RG2), and M-score(RG3), respectively, can be calculated according to formula B, with difference that the ROI was positioned in the liquor, instead of an artefact-free site.

Preferably, formula A is used when MRI outputs are obtained from an MR apparatus set on a magnetic field strength of 0.035 to 0.5 Tesla (i.e. low field strength), whereas formula B is preferably used when MRI outputs are obtained from an MR apparatus set on a magnetic field strength of 1.0 to 1.5 Tesla (i.e. high field strength).

In case of mid-field (i.e. 0.5-1.0 Tesla), either formula A or formula B can be used.

Preferably, in order to increase the statistical significance of the M-score mean value of reference groups, a minimum of 20 MRI outputs for the each reference group are collected and analysed.

On average, a number of 30 to 100 MRI outputs for the each reference group gives a good statistical significance.

In step III) of the method, the recorded lumbar spine MRI output of a subject of interest (‘SOI’) is examined, the examination being performed similarly to the analysis of the MRI outputs of the Reference Groups, i.e. by analysing the sagittal T1-weighted spin-echo sequence in vertebrae from L1 to L4, thus obtaining the relevant SNR_{L1-L4} (‘SOI-SNR_{L1-L4}’) or SQR_{L1-L4} (‘SOI-SQR_{L1-L4}’).

Again, also the lumbar spine MRI output of SOI is a lumbar spine MRI output obtained from a subject medical investigation, irrespective of the reason underlying the initial prescription of MR, and recorded in digital folders along with other medical investigation outputs of the same SOI. A medical workflow management system supports and assists in storing, sharing and providing said recorded MRI output for the purposes of examining the same. Advantageously, this means that also recorded lumbar spine MRI output of step III) is a document already available and retrieved from radiology archives of a clinic or a hospital.

Preferably, the MRI of SOI is obtained from the same MR apparatus model as used for the MRI outputs of step I). Similarly to what above discussed with respect to step I), this provision allows to analyse MRI outputs homogeneously obtained under the instrumental point of view. Actually, the MRI of SOI can be obtained from different MR apparatus model however, understandably, if the same model is used, the statistically precision rate of the subsequent comparison with the Reference Groups increases accordingly.

More preferably, the lumbar spine MRI output of SOI and the MRI outputs of step I) are obtained from exactly the same MR apparatus. This means that all the lumbar spine MRI outputs are homogeneously obtained by using the same instrument, thus maximising the overall precision of the method.

In step IV) of the method, the M-score of the subject of interest is calculated with respect to each Reference Group of step I), according to the following formulas:

$$\begin{matrix}M ‐ score ( 1 ) = [ ( S O I - S N {R}_{L 1 - L 4} ) - S N {R}_{R E F} ( R G 1 ) ] / {SD}_{R E F} ( R G 1 ) \\ M ‐ score ( 2 ) = [ ( S OI - SN {R}_{L 1 - L 4} ) - S N {R}_{R E F} ( R G 2 ) ] / {SD}_{R E F} ( R G 2 ) \\ M ‐ score ( 3 ) = [ ( S OI - SN {R}_{L 1 - L 4} ) - S N {R}_{R E F} ( R G 3 ) ] / {SD}_{R E F} ( R G 3 )\end{matrix}$$

$$or$$

$$\begin{matrix}M ‐ score ( 1 ) = [ ( S OI - {SQR}_{L 1 - L 4} ) - S Q {R}_{R E F} ( R G 1 ) ] / {SD}_{R E F} ( R G 1 ) \\ M ‐ score ( 2 ) = [ ( S OI - {SQR}_{L 1 - L 4} ) - S Q {R}_{R E F} ( R G 2 ) ] / {SD}_{R E F} ( R G 2 ) \\ M ‐ score ( 3 ) = [ ( S OI - {SQR}_{L 1 - L 4} ) - S Q {R}_{R E F} ( R G 3 ) ] / {SD}_{R E F} ( R G 3 )\end{matrix}$$

This means that each subject under examination receives three different M-scores to be used in the subsequent step of osteoporosis risk assessment.

In step V) of the method, the resulting three M-scores of step IV) are compared with the M-score mean value of the respective Reference Group resulting from step II), thus assessing for the subject of interest the possible risk of developing osteoporosis or directly diagnosing ongoing osteoporosis, based on recorded SOI's lumbar spine MRI output.

As a matter of fact, the main advantage of the method of the invention is that the SOI is not requested to be subjected to any additional medical investigation or analysis, as the method operates on documents already of records from a clinic or a hospital where the SOI was treated for whatsoever previous reasons.

In more details, the difference between M-score (1) and M-score(RG1) is a computed indicator of the status of SOI with respect to the healthy population. To this end, it is assumed that a difference value lower than “+1” indicates a “healthy subject”, whereas a difference value equal to or higher than “+1” indicates a “non-healthy subject”.

The difference between M-score (2) and M-score(RG2) is a computed indicator of the status of SOI with respect to the age-related risk population. To this end, it is assumed that a difference value higher than “−1” indicates an “OP patient subject” (i.e. a subject at risk of developing osteoporosis or affected by osteoporosis), whereas a difference value equal to or lower than “−1” indicates a “non-OP patient subject”.

The difference between M-score (3) and M-score(RG3) is a computed indicator of the status of SOI with respect to the OP patients population. To this end, it is assumed that a difference value higher than “−1” indicates a “eOP patient subject” (i.e. a subject affected by established osteoporosis—shortly ‘eOP’—, thus featuring a fragility fracture), whereas a difference value equal to or lower than “−1” indicates a “non-eOP patient subject” (i.e. a subject featuring no fragility fracture/s).

The combination of these three computed indicators allows to figure out the overall medical status of the SOI with respect to osteoporosis.

Preferably, step V) of the method comprises a sub-step V-i) wherein the difference between M-score (1) and M-score(RG1) and the difference between M-score (2) and M-score(RG2) are considered and compared, thus resulting in a first indication for the SOI. In this regard, three combinations of computed indicators are possible, as follows:
1) “healthy subject” AND “non-OP patient subject”, i.e. the SOI features a satisfactory bone mineralization;
2) “non-healthy subject” AND “non-OP patient subject”, i.e. the SOI features osteopenia;
3) “non-healthy subject” AND “OP patient subject”, i.e. the SOI features osteoporosis.
[the fourth combination of “healthy subject” AND “OP patient subject” is not included as actually not feasible, because a SOI cannot be at the same time healthy and showing osteoporosis signs.]

Preferably, step V) of the method comprises a sub-step V-ii) wherein the difference between M-score (1) and M-score(RG1) and the difference between M-score (3) and M-score(RG3) are considered and compared, thus resulting in a second computed indication for the SOI.

In this regard, two combinations of computed indicators are possible, as follows:
1) “healthy subject” AND “non-eOP patient subject”, i.e. the SOI features a satisfactory bone mineralization and shows no signs of osteoporosis=>NEGATIVE
2) “non-healthy subject” AND “eOP patient subject”, i.e. the SOI is clearly affected by established osteoporosis=>POSITIVE
[the other combinations of indicators are not included as actually not feasible, because opposed indicators cannot be assessed for the same subject (e.g. a subject cannot be at the same time healthy and featuring established osteoporosis)]

In other words, the method of the present invention is a method of computing indicators of a SOI, starting from the sagittal T1-weighted spin-echo sequence in vertebrae from L1 to L4 shown on recorded SOI's lumbar spine MRI output, while performing the steps I)-IV) above, thus obtaining computed indicators to be compared in step V) with reference indicators for osteopenia, osteoporosis risk or established osteoporosis. This means that the method above comprising steps I) to V) represents a method of data processing that could be used in a subsequent method of prognosis or diagnosis of osteoporosis.

If, the method results in an indication of osteopenia or directly in an osteoporosis or established osteoporosis diagnosis, a corresponding alert can be sent to the SOI, so that the latter can be immediately made aware of his/her own condition and contact a specialist for further investigations and assistance.

In preferred embodiments, step V) of the method comprises both sub-step V-i) and sub-step V-ii), to be performed preferably in this order. These embodiments are particularly advantageous insofar as the combination of step V-i) and step V-ii) allows to compare the MRI signals of a SOI not only to those observed for the healthy population, but also to those observed for the age-related risk population and eOP patients population, thus resulting in a complete and consistent overview of the SOI condition. Actually, the MRI of a SOI is compared with the MRIs of the age-related risk population to assess whether there is an ongoing osteoporosis, and with the MRIs of the eOP patients population to assess whether there are fragility fracture/s, i.e. established osteoporosis.

The method of the invention is therefore a reliable, reproducible and sensitive method for predicting or diagnosing osteoporosis from magnetic resonance images, through a simple and quick statistical processing, i.e. by only statistically correlating MRI of a subject to MRI of reference groups, independently of other standard techniques such as, for example, MOC, QCT, DXA, or clinical diagnosis of osteoporosis.

In consideration of what above, it is clear that the method of the invention features several advantages and benefits under many points of view.

First, any patient who undergoes magnetic resonance imaging of the lumbar spine for reasons independent of osteoporosis, could anyway receive diagnostic information also on his/her medical status concerning osteoporosis, without additional investigations or tests and without the use of ionizing radiation, regardless of previous suspicion of osteoporotic pathology.

This is very precious and desirable as, in this way, it is possible to early identify patients at risk of osteoporosis or with the pathology already in progress, notwithstanding the asymptomatic nature of osteoporosis.

Moreover, the overall method of the invention is very convenient insofar as neither the patient nor the national health system incur additional costs.

Therefore, those directly benefiting of the method of the invention are:
patients at risk of developing osteoporosis or with ongoing osteoporosis, who are completely unaware of their situation. These patients, duly alerted by the method above described, can timely start the treatment and therapeutic-preventive indications of the case with their medical doctor;
national health systems: prediction and/or early diagnosis is based on magnetic resonance tests already of records, for which there is no additional cost. Moreover, early diagnosis allows to significantly reduce the incidence of fractures ascribable to osteoporosis and consequently reduce the relative high cost of managing the same, this amounting to a considerable saving in overall healthcare costs for the national health system and for the patients themselves;
public, private or accredited private radiology services with magnetic resonance equipment: without additional costs or added machine time compared to the standard exam, they can provide the patient with information about the osteoporotic pathology, irrespective of the reason underlying the prescription of MR.

Given the high number of magnetic resonance investigations that are performed every day for various clinical indications independent of osteoporosis, the method of the invention advantageously allows to identify a far larger number of patients at risk of osteoporosis or with the pathology ongoing, than classic CBM investigation, that will be able to access adequate prevention or treatment measures.

Another advantage is that the method of the invention can be applied to any MRI equipment, regardless of suppliers or magnetic field strength, as well as to any data storage and processing system, such as those mentioned above, e.g. PACS, RIS or DICOM. Also, there are no exclusion criteria for patients, as the method is universally applicable to any MRI output.

It is also clear that the reference database and accordingly the three Reference Groups enjoy a continuous upload of MRI outputs during the day-by-day clinical practice, so that the precision of the resulting method outcome, i.e. prediction and/or diagnosis of osteoporosis, increases consequently and advantageously overtime. In other words, the more the method is implemented, the better the potential osteoporosis patients are alerted.

The present invention also relates to a computer program comprising instructions which, when the program is executed by a computer, cause the computer to carry out the steps of the method of the invention.

Moreover, the present invention relates to a computer-readable data carrier having stored thereon said computer program.

Additionally, the present invention concerns a computer-implemented system for executing the above method.

In particular, said computer-implemented system comprises:
a) at least one digital repository in communication with at least one medical workflow management system of a clinic or a hospital, the repository being for archiving the recorded MRI outputs received from said at least one medical workflow management system, wherein the MRI outputs are made anonymous via software before being archived in a reference database, as per step I) of the method above,
b) a software platform operating on said reference database and comprising a computer program designed to perform the calculations, examination and comparison of steps II) to V) of the method above, and
c) a plurality of users' computing devices for access to said software platform, so as to send inquiries and receive reports, each user being associated a digital identity comprising authentication means, wherein said authentication means comprise a processor capable of automatically generating an authentication code in response to an interrogation for validating the digital identity of a user.

A “digital repository” is any medium or digital system capable of storing digital information. Examples of suitable digital repositories are a hard disk, a cloud server, a fog, a USB key, etc. Preferably, the digital repository of the present invention is a cloud server.

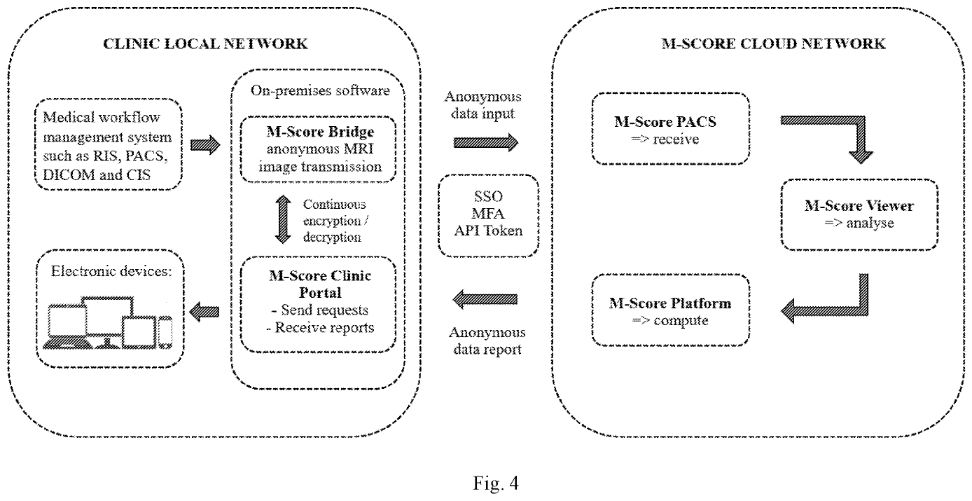

With reference to FIG. 4, a preferred configuration of the system of the invention is implemented. This system is cloud-based and included the following technical features.

### M-Score PACS

“M-Score PACS” is the name of a software instance. In cloud computing, an instance is a server resource provided by third-party cloud services. The software has been customized to be able to integrate with the M-Score Viewer and to offer its users information relating to the M-Score method (SNR value, SNRL, SD, etc.).

The main application, developed mainly in PHP with system services that manage DICOM, is installed on an AWS EC2 Windows Server machine. Image storage, instead, is managed by the AWS FsX for Windows service.

The reference database is a PostgresSQL on AWS RDS.

### M-Score Viewer

“M-Score Viewer” is the name of a DICOM viewer customized to be able to extract signal intensity values on MR images in a guided manner.

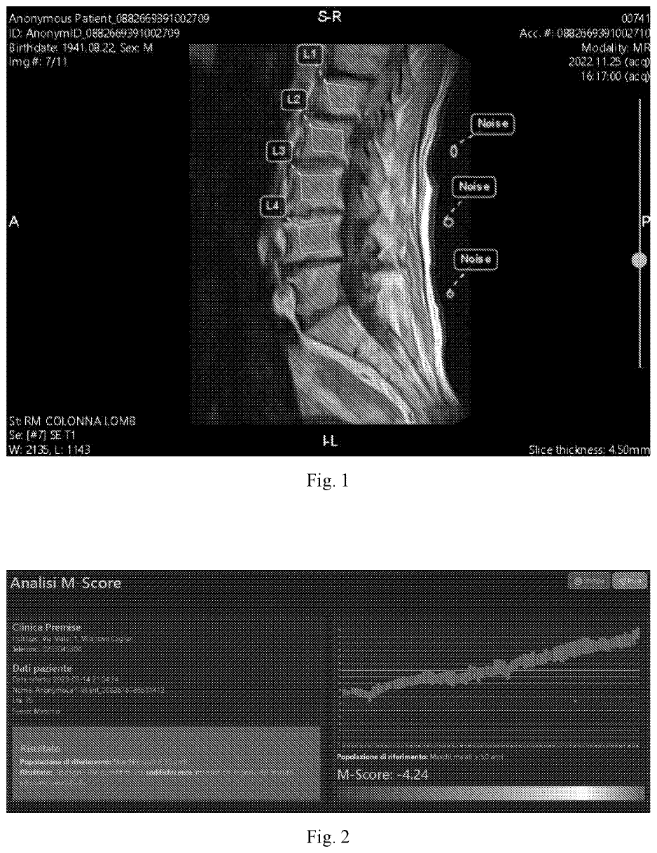

It is possible to select ROIs on the areas of interest, extract the data and calculate the SNR or SNRL value, as shown in FIG. 1.

### M-Score-Platform

“M-Score-Platform” is the name of an application back-end implemented by using the Laravel framework supported by JetStream, responsible for generating an assessment of bone health based on graphical analysis of MR images (M-Score Viewer), and statistical analysis by using aggregate anonymized data of groups of patients, called reference populations (M-Score Platform). M-Score-Platform operates solely with anonymized data and manages users, reference populations, requests and results of each clinic or hospital. The clinics or hospitals have the opportunity to access a reserved area where they can obtain the API keys*, configure their company data and check the quantity of credits available to make requests and possibly obtain the details for the purchase of new credits. [* an API key is an alphanumeric string that API developers use to control access to their APIs. An API is a communication mechanism that allows data exchange between two software modules. Once you create an API for your module, other application developers can call your API to integrate your functionality into their code.]

Alternatively, Single sign-on (SSO) is all about users gaining access to different apps with a single authentication. As for multifactor authentication (MFA), it adds a layer of security upon authentication to verify the user's identity.

The application included an administration section accessible only to one or more administrators, which contained a management panel for users (and therefore for registered clinics/hospitals), reference populations, requests and bone health reports.

The administration panel of requests allowed the administrator to take measurements via M-Score Viewer and then determine and save in the PACS the values used for generating reference populations and bone health reports.

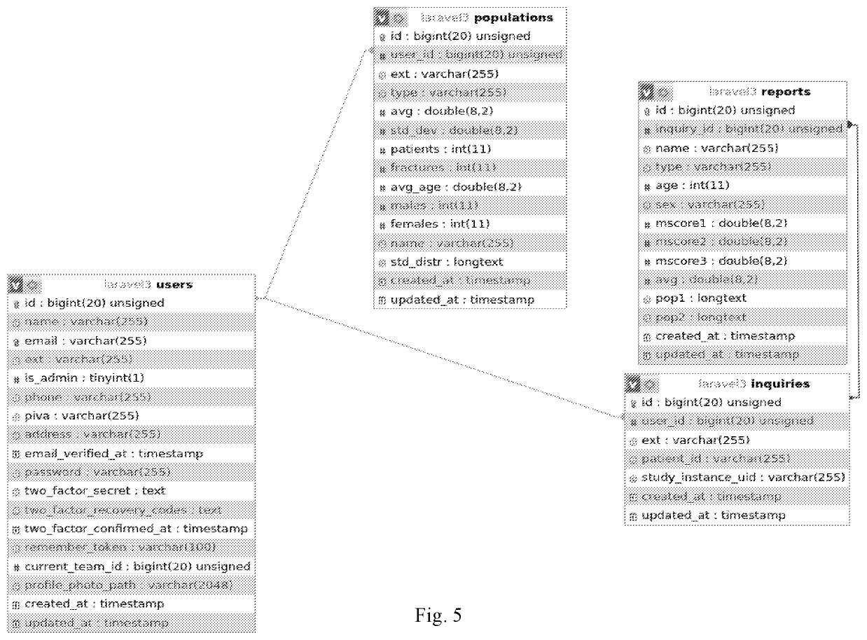

The data are organized in the database as exemplary illustrated in FIG. 5.

The “users” are the clinics/hospitals registered for the service. Users can be created via registration forms. At a later time, the administrator can associate a specific user with a specific unique identifier of the clinic/hospital, so as to be able to associate the data present in the PACS with the data present in the application database.

The “populations” are aggregate statistical data of populations used as reference, as referred above as “Reference Groups”. Reference groups or populations are generated by an administrator using data from clinic/hospital patients whose bone density have already been assessed, with which it is possible to compare the data of the patient requesting the report.

The “inquiries” are the requests submitted by the clinics/hospitals, concerning their patients.

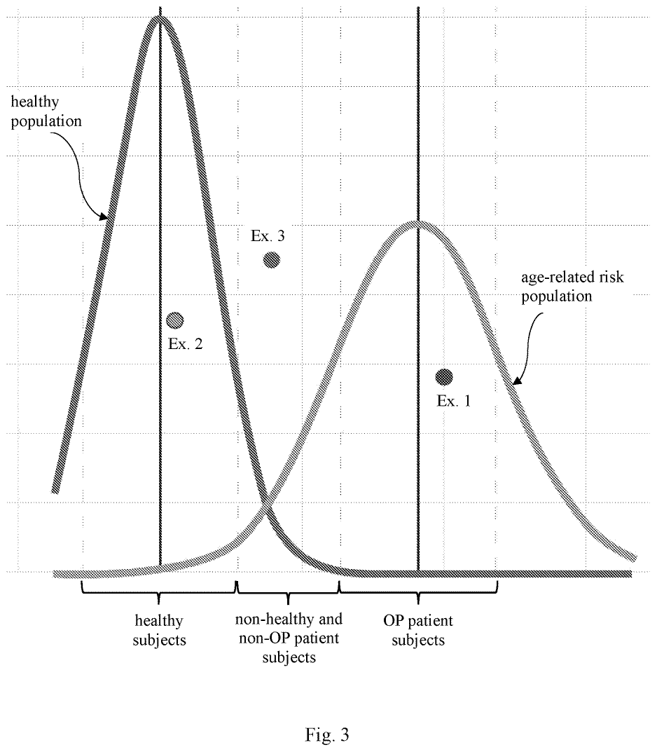

The “reports” are bone health reports ready to be delivered. The reports have a graphical appearance similar to what depicted in FIG. 3.

Administrators have the following sections available:
1. User administration
2. Administration of reference populations
3. Administration requests

Users have the following sections available:
1. User administration (personal information, security options, password change)
2. API key management
3. Credit management

### M-Score Bridge

“M-Score Bridge” is the name of a set of dedicated software to allow Q/R on the clinics/hospitals PACS to identify and receive the relevant tests and anonymize the patient data and deanonymize the report data received from the M-Score Clinic Portal from M-Score Platform.

### M-Score Clinic Portal

“M-Score Clinic Portal” is the name of a local client application designed to reside on one or more clinic/hospital computers and be used by staff to request bone health reports and download results in PDF format, when available. This application communicates with M-Score-Platform via API (the API key obtained in the user section of M-Score Platform must be entered into the M-Score Clinic Portal application).

The application have at least 3 navigable sections:
1. API key configuration
2. Search exam
3. Request list

### Request List

The user can view the list of existing requests and their status. The data that populates this section come from the M-Score-Platform application database, in particular from the requests and results tables, and was in anonymized form. To these is added the name and surname pair obtained during the search phase and saved when creating a request.

If there was a result related to a request, a “generate” button allowed to generate and save the bone health report in PDF format. If there was no result related to a particular request, the latter resulted to be “in progress”.

Results If a request has already been processed, the related bone health report can be downloaded.

The report is transmitted from M-Score-Platform in JSON format containing the anonymized data of a record in the results table. M-Score Clinic Portal takes care of de-anonymizing the report using M-Score Bridge and finally generating a PDF file containing the report that the user can download by any electronic device.

It should be understood that the individual preferred aspects described hereabove for the preferred embodiment of the system, as exemplary depicted in FIG. 4, should likewise be considered as individually preferred also for the method and system of the invention.

It should be also understood that the method and system as above described are not limited to lumbar spine area, being indeed similarly applicable to other skeletal segments simply by selecting ROIs on different anatomical areas to identify the signal intensity and use the noise to obtain the signal/noise ratio.

Below is a non limiting list of alternative areas to segment with the related ROIs for each skeletal segment on T1 MRI sequences.

### Hip and Femur

Femoral Neck: A critical area for evaluating osteoporosis, the femoral neck can be analysed by segmenting an area that begins at the base of the femoral neck and extends to the proximal metaphysis of the femur. It is important to include the cortical and internal trabecular bone, while avoiding areas with marked presence of soft tissue.

Greater Trochanter: Another relevant area is the greater trochanter, a region that shows significant changes in bone density in cases of osteoporosis. The ROI here includes the internal trabecular portion, while avoiding the insertion of the muscles and tendons to minimize interference.

Data obtained—SNR_{GT-CF}

### Wrist and Hand

Distal Radius: In the distal part of the radius, it is useful to segment the area between the styloid process and the metaphysis. This region is indicative of changes in bone density, especially in conditions of osteopenia and osteoporosis.

Carpal Bones: Although less common, the carpal bones can be examined for specific bone density losses. An ROI can be defined by including the scaphoid and lunate bones as representative of the bone health of the wrist.

Data obtained—SNR_{OS-RD}

### Cervical and Thoracic Vertebrae

Cervical and Dorsal Vertebral Bodies: Similar to the lumbar spine, the cervical and dorsal vertebrae can be examined by segmenting the vertebral bodies. ROIs are delineated including the central portions of the vertebral bodies, from C3 to D12, focusing on the internal trabecular bone to evaluate the presence of osteoporotic changes.

Data obtained—SNR_{C3-C7} and SNR_{D6-D12}

### Knee

Tibial Plateau: A critical area for osteoporotic analysis is the tibial plateau, which can be divided into two main ROIs: the medial portion and the lateral portion. Segmentation of these areas should include the subchondral trabecular bone, focusing on any changes in bone density and trabecular structure.

Proximal Tibial Metaphysis: This area, right below the tibial plateau, is important for evaluating the transition between the cortical bone and the trabecular bone, which may be affected by osteoporosis.

Femoral Condyles: The medial and lateral femoral condyles are critical for the evaluation of osteoporosis in the knee. The ROI in these areas includes the subchondral trabecular bone and adjacent areas, to monitor bone density and identify any structural changes indicative of osteoporosis.

Distal Femoral Metaphysis: Similar to the tibia, this region is also important for the evaluation of osteoporosis. Segmentation should cover a significant portion of the metaphyseal trabecular bone, examining variations in density and structure.

Data obtained—SNR_{CF-PT}

### Shoulder

Central Area of the Humeral Head: This area is fundamental for evaluating changes in bone density and microarchitecture associated with osteoporosis. The ROI should focus on the trabecular bone, with particular attention to changes in trabecular density and configuration beneath the bone cortex.

Anatomic Neck of the Humerus: The area around the anatomical neck, where the head of the humerus joins the body of the bone, is crucial for evaluating osteoporosis. Segmentation in this region should include trabecular bone to monitor potential osteoporotic rarefactions.

Data obtained—SNR_{CO-TO}

### Elbow

Lateral Epicondyle of the Humerus: Area commonly involved in overuse injuries, such as lateral epicondylitis (“tennis elbow”). The analysis of this area can help detecting early changes in bone density or signs of repeated microtrauma.

Medial Epicondyle of the Humerus: Important for identifying medial epicondylitis (“golfer's elbow”) and other conditions that affect the tendons and bony attachment in this region.

Olecranon of the Ulna: The olecranon is the leverage point for the extensor muscles of the forearm and may be subject to fractures or other traumatic pathologies. It is a critical region for evaluating post-traumatic bone integrity.

Head of the Ulna: The head of the ulna forms part of the distal radioulnar joint, which may be affected by osteoarthritis or other degenerative pathologies.

Radial Head: The radial head plays a crucial role in the elbow joint and rotational motion of the forearm. Fractures and degenerative changes in this area are common and can be diagnosed by analyzing MRI images.

Data obtained—SNR_{EO-RU}

### Ankle and Foot

Metaphysis and Tibial Epiphysis: These areas are particularly vulnerable to stress fractures, impact fractures, and degenerative changes. Analysing the density and structural integrity in these areas can reveal the presence of osteoporosis, osteoarthritis or other bone diseases.

Tibial Malleolus: A critical region involved in the formation of the ankle joint. Injuries in this area often occur following twisting or direct impact trauma, such as in ankle sprains.

Lateral malleolus: It is less involved in load transfer than the medial malleolus, but is very susceptible to fractures due to trauma or twisting of the foot. This area should be examined for evaluation of fractures and post-traumatic conditions.

Calcaneal Body: As the bone that forms the heel, the calcaneus is essential for absorbing impacts during walking. Calcaneal fractures can be complex and require careful analysis to identify fracture patterns and plan surgery or conservative treatments.

Data obtained—SNR_{MT-CC}

Depending on the skeletal segment selected, the method and the system of the invention can be successfully applied, thus allowing to achieve a reliable and useful result on the risk of developing osteoporosis (i.e. osteopenia) or on assessment of ongoing osteoporosis, as well as on assessment of established osteoporosis.

Below are working examples of the present invention provided for illustrative purposes.

### EXAMPLES

### Example 1

The method of the invention has been implemented at “Centro di Medicina Preventiva” of Rozzano (IT), a private medical centre specialized in diagnostic imaging investigations.

In the system PACS, with interface viewer OsiriX, 2′000 MRI outputs, all being obtained from the same MR apparatus, i.e. S-SCAN (0.25 Tesla field strength) supplied by ESAOTE SpA (IT), were collected starting from July 2014 to date.

Among the 2′000 MRI outputs, those compliant with the criteria of the three Reference Groups are selected and classified accordingly, thus resulting in the following:
RG1—healthy population—70 MRI outputs
RG2—age-related risk population—90 MRI outputs
RG3—OP patients population—30 MRI outputs (whose patients have more than 50 years)

For all the MRI outputs, a sagittal T1-weighted spin-echo sequence, while segmenting vertebral bodies from L1 to L4, as shown in FIG. 1. A region of interest (ROI) was placed in the vertebral body excluding cortical bone, subchondral abnormalities, focal lesions (e.g., hemangiomas), and posterior venous plexus. Three ROIs were used for each vertebra, each of them acquired on a different slice, with their mean used for analysis. A ROI was also positioned in an artefact-free site outside the patient to measure the noise. Signal-to-noise ratio (SNR) was obtained by dividing the intra-vertebral signal intensity by the standard deviation of the noise. The positioning of the ROIs can be done manually by an operator.

The SNR_{L1-L4} was measured in all MRI outputs of the each reference group, and standard deviation (SD_{REF}), resulting as follows:

$$\begin{matrix}RG 1 \text{=>} {SNR}_{L 1 - L 4} = 39.88 - S D = 6.13 \\ RG 2 \text{=>} {SNR}_{L 1 - L 4} = 69. 10 - S D = 10.04 \\ RG 3 \text{=>} {SNR}_{L 1 - L 4} = 76.61 - S D = 9.77\end{matrix}$$

The M-score for each MRI output was calculated, as per step II) of the method. An exemplary visual depiction of the calculation of M-score for a subject of RG2 is provided in FIG. 2.

Subsequently, the M-score mean value for each Reference Group is calculated according to the formulas provided above, for step II) of the method: Once the Reference Groups have been duly classified and analysed, the MRI output of the subject of interest, anonymously examined, has been considered.

The resulting SOI-SNR_{L1-L4}=75.00 was used to calculate M-score (1), M-score (2), and M-score (3), according to the formulas above reported for step IV) of the method.

These three SOI M-scores have been compared with the M-score mean value of each Reference Group, as per step V) of the method.

$$\begin{matrix}M ‐ score ( 1 ) = ( 75. - 39. 88 ) / 6.13 = 5.73 \\ M ‐ score ( 2 ) = ( 75. - 69. 10 ) / 10.04 = 0.59 \\ M ‐ score ( 3 ) = ( 75. - 76. 61 ) / 9.77 = - 0.16\end{matrix}$$

The outcome of the method on the subject of interest gave the following information: Result of Step V-i): combination 3) “non-healthy subject” AND “OP patient subject”, i.e. the SOI features osteoporosis

Result of Step V-ii): POSITIVE, i.e. the SOI is clearly affected by established osteoporosis In this case, the SOI was diagnosed with an ongoing established osteoporosis and thus immediately alerted to contact a specialist for further assistance.

### Example 2

The same Reference Groups of Example 1 were kept, whereas the MRI output of another subject of interest, anonymously examined, has been considered.

The resulting SOI-SNR_{L1-L4}=42.00 was used to calculate M-score (1), M-score (2), and M-score (3), according to the formulas above reported for step IV) of the method.

These three SOI M-scores have been compared with the M-score mean value of each Reference Group, as per step V) of the method.

$$\begin{matrix}M ‐ score ( 1 ) = ( 42. - 39. 88 ) / 6.13 = 0.34 \\ M ‐ score ( 2 ) = ( 42. - 69. 10 ) / 10.04 = - 2.70 \\ M ‐ score ( 3 ) = ( 42. - 76. 61 ) / 9.77 = - 3.54\end{matrix}$$

The outcome of the method on the subject of interest gave the following information:
Result of Step V-i): combination 1) “healthy subject” AND “non-OP patient subject”, i.e. the SOI features a satisfactory bone mineralization and shows no risks of developing osteoporosis
Result of Step V-ii): NEGATIVE

In this case, the SOI is a healthy subject.

### Example 3

The same Reference Groups of Example 1 were kept, whereas the MRI output of another subject of interest, anonymously examined, has been considered.

The resulting SOI-SNR_{L1-L4}=58.50 was used to calculate M-score (1), M-score (2), and M-score (3), according to the formulas above reported for step IV) of the method.

These three SOI M-scores have been compared with the M-score mean value of each Reference Group, as per step V) of the method.

$$\begin{matrix}M ‐ score ( 1 ) = ( 58.5 - 39. 88 ) / 6.13 = 3. \\ M ‐ score ( 2 ) = ( 58.5 - 69. 10 ) / 10.04 = - 1.06 \\ M ‐ score ( 3 ) = ( 58.5 - 76. 61 ) / 9.77 = - 1.85\end{matrix}$$

The outcome of the method on the subject of interest gave the following information:
Result of Step V-i): combination 2) “non-healthy subject” AND “non-OP patient subject”, i.e. the SOI features osteopenia, which could be potentially predictive of developing osteoporosis
Result of Step V-ii): NEGATIVE In this case, the SOI features osteopenia, so that a corresponding alert was sent.

In FIG. 3, the depiction of the condition above detected after step V-i) for the subjects of interest of Examples 1-3 is shown. Particularly, the curves concerning RG1 and RG2 are reported, with respect to which the resulting outcomes of the SOIs are located. It is worthwhile to be noted that even only this sub-step alone is sufficient for assessing the condition of a SOI with respect to osteoporosis from a simple MRI analysis. In this regard, it should be reminded that for the first time the method of the invention has allowed to diagnosis osteoporosis, and also established osteoporosis, by relying on MRI outputs already available, i.e. without requiring additional and expensive investigations or tests and without the use of ionizing radiation, regardless of previous suspicion of osteoporotic pathology.

### Example 4

With reference to FIG. 4, a preferred configuration of the system of the invention was implemented.

The system was cloud-based and included the following technical features.

### M-Score PACS

M-Score PACS was a software instance. In cloud computing, an instance is a server resource provided by third-party cloud services. The software has been customized to be able to integrate with the M-Score Viewer and to offer its users information relating to the M-Score method (SNR value, SNRL, SD, etc.).

The main application, developed mainly in PHP with system services that manage DICOM, was installed on an AWS EC2 Windows Server machine. Image storage, instead, was managed by the AWS FsX for Windows service.

The reference database is a PostgresSQL on AWS RDS.

### M-Score Viewer

It was a DICOM viewer customized to be able to extract signal intensity values on MR images in a guided manner.

It was possible to select ROIs on the areas of interest, extract the data and calculate the SNR or SNRL value, as shown in FIG. 1.

### M-Score-Platform

M-Score-Platform (application back-end) was implemented by using the Laravel framework supported by JetStream, responsible for generating an assessment of bone health based on graphical analysis of MR images (M-Score Viewer), and statistical analysis by using aggregate anonymized data of groups of patients, called reference populations (M-Score Platform). M-Score-Platform operated solely with anonymized data and manages users, reference populations, requests and results of each clinic or hospital. The clinics or hospitals had the opportunity to access a reserved area where they can obtain the API keys, configure their company data and check the quantity of credits available to make requests and possibly obtain the details for the purchase of new credits.

The application included an administration section accessible only to one or more administrators, which contained a management panel for users (and therefore for registered clinics/hospitals), reference populations, requests and bone health reports.

The administration panel of requests allowed the administrator to take measurements via M-Score Viewer and then determine and save in the PACS the values used for generating reference populations and bone health reports.

The data were organized in the database as exemplary illustrated in FIG. 5.

The “users” are the clinics/hospitals registered for the service. Users can be created via registration forms, there including data such as:
1. id user
2. name
3. email
4. email verified at
5. password
6. ext (PACS id)
7. is_admin (0 user, 1 admin)
8. phone number

At a later time, the administrator can associate a specific user with a specific unique identifier of the clinic/hospital, so as to be able to associate the data present in the PACS with the data present in the application database.

The “populations” were aggregate statistical data of populations used as reference, as referred above as “Reference Groups”. Reference groups or populations were generated by an administrator using data from clinic/hospital patients whose bone density had already been assessed, with which it was possible to compare the data of the patient requesting the report.

The “inquiries” were the requests submitted by the clinics/hospitals, concerning their patients.

The “reports” were bone health reports ready to be delivered. The reports have a graphical appearance similar to what depicted in FIG. 3.

Administrators have the following sections available:
1. User administration
2. Administration of reference populations
3. Administration requests

Users have the following sections available:
1. User administration (personal information, security options, password change)
2. API key management
3. Credit management

### M-Score Bridge

M-Score Bridge was a set of dedicated software to allow Q/R on the clinics/hospitals PACS to identify and receive the relevant tests and anonymize the patient data and deanonymize the report data received from the M-Score Clinic Portal from M-Score Platform.

### M-Score Clinic Portal

M-Score Clinic Portal (local client application) was designed to reside on one or more clinic/hospital computers and be used by staff to request bone health reports and download results in PDF format, when available. This application communicated with M-Score-Platform via API (the API key obtained in the user section of M-Score Platform must be entered into the M-Score Clinic Portal application).

The application has 3 navigable sections:
1. API key configuration
2. Search exam
3. Request list

### Request List

The user can view the list of existing requests and their status. The data that populates this section come from the M-Score-Platform application database, in particular from the requests and results tables, and was in anonymized form. To these is added the name and surname pair obtained during the search phase and saved when creating a request.

If there was a result related to a request, a “generate” button allowed to generate and save the bone health report in PDF format. If there was no result related to a particular request, the latter resulted to be “in progress”.

### Results

If a request had already been processed, the related bone health report can be downloaded. The report is transmitted from M-Score-Platform in JSON format containing the anonymized data of a record in the results table. M-Score Clinic Portal takes care of de-anonymizing the report using M-Score Bridge and finally generating a PDF file containing the report that the user can download by any electronic device.

1. A computer-implemented method of predicting and diagnosing osteoporosis on the basis of lumbar magnetic resonance imaging techniques, the method comprising the steps of:
I) collecting recorded lumbar spine MRI outputs, obtained from Magnetic Resonance apparatus, by means of a medical workflow management system selected from RIS, PACS, DICOM and CIS, and archiving to create a reference database, wherein said lumbar spine MRI outputs are classified into three different reference groups, according to the following criteria:
Reference Group 1 (RG1)—MRI outputs of subjects aged 20-30 years
Reference Group 2 (RG2)—MRI outputs of subjects aged higher than 75 years
Reference Group 3 (RG3)—MRI outputs of subjects that have received a diagnosis of established osteoporosis, after fragility fracture detection within the lumbar spine area,
II) calculating a the M-score for each MRI output of step I), according to the formula:
$$M ‐ score = ( SNRL 1 ‐ L 4 - SNRREF ) / SDREF \qquad ( A )$$
$$or$$
$$M ‐ score = ( SQRL 1 ‐ L 4 - SQRREF ) / SDREF \qquad ( B )$$
wherein
SNRL1-L4 denotes the signal-to-noise ratio obtained by dividing the intra-vertebral signal intensity by the standard deviation of the noise in vertebrae from L1 to L4 within the sagittal T1-weighted spin-echo sequence, of the MRI output,
SNRREF denotes the SNR mean value of the Reference Group to which the MRI output belongs,
SQRL1-L4 denotes the signal-to-liquor ratio obtained by dividing the intra-vertebral signal intensity by the standard deviation of the liquor in vertebrae from L1 to L4 within the sagittal T1-weighted spin-echo sequence, of the MRI output,
SQRREF denotes the SQR mean value of the Reference Group to which the MRI output belongs, and
SDREF denotes the standard deviation of the Reference Group to which the MRI output belongs,
and then calculating the M-score mean value for each Reference Group of step I), named M-score(RG1), M-score(RG2), and M-score(RG3) respectively,
III) examining the recorded lumbar spine MRI output of a subject of interest (‘SOI’), by analysing the sagittal T1-weighted spin-echo sequence in vertebrae from L1 to L4, thus obtaining the relevant SNRL1-L4 (‘SOI-SNRL1-L4’) or SQRL1-L4 (‘SOI-SQRL1-L4’),
IV) calculating the M-score of the subject of interest with respect to each Reference Group of step I), according to the following formulas:
$$\begin{matrix}M ‐ score ( 1 ) = [ ( S O I ‐ SNRL 1 ‐ L 4 ) - S N R R E F ( R G 1 ) ] / SDREF ( R G 1 ) \\ M ‐ score ( 2 ) = [ ( S O I ‐ SNRL 1 ‐ L 4 ) - S N R R E F ( R G 2 ) ] / SDREF ( R G 2 ) \\ M ‐ score ( 3 ) = [ ( S O I ‐ SNRL 1 ‐ L 4 ) - S N R R E F ( R G 3 ) ] / SDRE F ( RG 3 )\end{matrix}$$
$$or$$
$$\begin{matrix}M ‐ score ( 1 ) = [ ( S O I ‐ SQRL 1 ‐ L 4 ) - S Q R R E F ( R G 1 ) ] / SDREF ( R G 1 ) \\ M ‐ score ( 2 ) = [ ( S O I ‐ SQRL 1 ‐ L 4 ) - S Q R R E F ( R G 2 ) ] / SDREF ( R G 2 ) \\ M ‐ score ( 3 ) = [ ( S O I ‐ SQRL 1 ‐ L 4 ) - S Q R R E F ( R G 3 ) ] / SDREF ( R G 3 )\end{matrix}$$
and
V) comparing the resulting three M-scores of step IV) with the M-score mean value of the respective Reference Group resulting from step II), thus assessing for the subject of interest the possible risk of developing osteoporosis or diagnosing osteoporosis.

2. The method of claim 1, wherein the steps I)-V) are implemented by a computer executing a computer program comprising instructions, which cause the computer to carry out said steps.

3. The method of claim 1, wherein the lumbar spine MRI outputs of step I) are obtained from the same MR apparatus model.

4. The method of claim 1, wherein a minimum of 20 MRI outputs for the each reference group are collected and analysed.

5. The method of claim 4, wherein a number of 30 to 100 MRI outputs for the each reference group is collected and analysed.

6. The method of claim 1, wherein, in step III), the MRI of SOI is obtained from the same MR apparatus model as used for the MRI outputs of step I).

7. The method of claim 6, wherein the MRI output of SOI of step III) and the MRI outputs of step I) are obtained from exactly the same MR apparatus.

8. The method of claim 1, wherein, in step V):
the difference between M-score (1) and M-score(RG1) is an indicator of the status of SOI with respect to the healthy population, and a difference value lower than “+1” indicates a “healthy subject”, whereas a difference value equal to or higher than “+1” indicates a “non-healthy subject”;
the difference between M-score (2) and M-score(RG2) is an indicator of the status of SOI with respect to the age-related risk population, and a difference value higher than “−1” indicates an “OP patient subject”, whereas a difference value equal to or lower than “−1” indicates a “non-OP patient subject”; and
the difference between M-score (3) and M-score(RG3) is an indicator of the status of SOI with respect to the OP patients population, and a difference value higher than “−1” indicates a “eOP patient subject”, whereas a difference value equal to or lower than “−1” indicates a “non-eOP patient subject”.

9. The method of claim 8, wherein step V) comprises a sub-step V-i), wherein the difference between M-score (1) and M-score(RG1) and the difference between M-score (2) and M-score(RG2) are considered and compared, thus resulting in the following three combinations of computed indicators:
1) “healthy subject” AND “non-OP patient subject”, i.e. the SOI features a satisfactory bone mineralization and shows no risks of developing osteoporosis;
2) “non-healthy subject” AND “non-OP patient subject”, i.e. the SOI features osteopenia, which could be potentially predictive of developing osteoporosis;
3) “non-healthy subject” AND “OP patient subject”, i.e. the SOI features osteoporosis, which is also deemed to be prognostic of fragility fracture/s.

10. The method of claim 8, wherein step V) comprises a sub-step V-ii) wherein the difference between M-score (1) and M-score(RG1) and the difference between M-score (3) and M-score(RG3) are considered and compared, thus resulting in the following two combinations of computed indicators:
1) “healthy subject” AND “non-eOP patient subject”, i.e. the SOI features a satisfactory bone mineralization and shows no signs of osteoporosis=>NEGATIVE
2) “non-healthy subject” AND “eOP patient subject”, i.e. the SOI is clearly affected by established osteoporosis=>POSITIVE

11. The method of claim 10, wherein step V) comprises both sub-step V-i) and sub-step V-ii), to be performed in this order.

12. A computer program comprising instructions which, when the program is executed by a computer, cause the computer to carry out the steps of the method of claim 1.

13. (canceled)

14. A computer-implemented system for executing the method of claim 1, the computer-implemented system comprising:
a) at least one digital repository in communication with at least one medical workflow management system of a clinic or a hospital, the repository being for archiving the recorded MRI outputs received from said at least one medical workflow management system, wherein the MRI outputs are made anonymous via software before being archived in a reference database, as per step I) of the method,
b) a software platform operating on said reference database and comprising a computer program designed to perform the calculations, examination and comparison of steps II) to V) of the method, and
c) a plurality of users' computing devices for access to said software platform, so as to send inquiries and receive reports, each user being associated a digital identity comprising authentication means, wherein said authentication means comprise a processor capable of automatically generating an authentication code in response to an interrogation for validating the digital identity of a user.

15. The computer-implemented system of claim 14, the system being cloud-based.
